(19) United States
(12) Patent Application Publication
(10) Pub. No.: US 2024/0271074 A1
(43) Pub. Date: Aug. 15, 2024

(54) FED-BATCH FERMENTATION PROCESS

(71) Applicant: DSM IP ASSETS B.V., Heerlen (NL)

(72) Inventor: Wouter Adrianus VAN WINDEN, Echt (NL)

(21) Appl. No.: 18/563,134

(22) PCT Filed: May 24, 2022
(86) PCT No.: PCT/EP2022/064059

§ 371 Date: Nov. 21, 2023

(30) Foreign Application Priority Data
May 25, 2021 (EP) 21175746.3

(51) Int. Cl. C12M 1/00; C12M 1/36

(52) U.S. Cl. CPC C12M 23/58; C12M 41/48

(57) ABSTRACT

The present invention relates to a method for cultivating a microorganism, comprising the steps of
(i) adding into a first bioreactor a medium and an inoculum comprising the microorganism and producing a fermentation broth in fed-batch culture; and
(ii) adding into a second bioreactor a second medium and part of the fermentation broth from step (i) and producing a second fermentation broth in a second fed-batch culture; and
(iii) adding into a next bioreactor a next medium and a part of the fermentation broth from step (ii), and producing a next fermentation broth in a next fed-batch culture,
   wherein a feed is introduced into the first, second and next bioreactor.

### FIELD OF THE INVENTION

The present invention relates to a method for cultivating a microorganism. According to a further aspect, the present invention relates to a fermentation plant comprising at least three bioreactors for carrying out the process as disclosed herein.

### BACKGROUND

Industrial fermentation plants can be mono-plants where many repeated batches of a single product are executed in multiple bioreactors, also known as fermentation vessels. When these industrial fermentation processes are executed in conventional fed-batch mode, they are characterized by a low initial and high final bioreactor filling. After a fed-batch fermentation process has been completed the bioreactor wherein the fed-batch fermentation took place is emptied, sterilized and a new fed-batch fermentation can be started by adding medium and introducing a fresh inoculum of a microorganism from a separate inoculum bioreactor. By consequence the average degree of filling of these fed-batch bioreactors often is low in comparison to processes run in batch and chemostat mode. Thus, while many fermentation plants are operated at maximum capacity, a significant part of the available fermentation capacity is filled with air rather than fermentation broth. This is a waste of capital invested. This problem is particularly present for large bioreactors, typically ranging from 30 to 500 m³.

WO2016/189203 discloses a method for producing a biosynthetic product in a cascade of bioreactors comprising a biomass production reactor and a product formation reactor, wherein part of the microorganism culture from the biomass production reactor is fed to the product formation reactor which contains nutrient depleted medium. The microbial culture that is produced in a product formation reactor may also be fed to a subsequent product formation reactor. Before feeding into the product formation reactor the microbial culture from either the biomass production or production formation reactor is concentrated.

There is a need in the art for improved methods for industrial fermentation processes.

### SUMMARY

The present invention relates to a method for cultivating a microorganism, comprising the steps of
(i) adding into a first bioreactor a medium and an inoculum comprising the microorganism and producing a fermentation broth in fed-batch culture; and
(ii) adding into a second bioreactor a second medium and part of the fermentation broth from step (i) and producing a second fermentation broth in a second fed-batch culture; and
(iii) adding into a next bioreactor a next medium and a part of the fermentation broth from step (ii), and producing a next fermentation broth in a next fed-batch culture, wherein a feed is introduced into the first, second and next bioreactor.

The invention also relates to a fermentation plant comprising a least three bioreactors which are connected with means for transporting fermentation broth between the bioreactors.

The invention further relates to a process for controlling the method as disclosed herein, comprising the steps of
(i) Determining a time the fermentation broth is present in the first, second and/or next bioreactor
(ii) Determining an amount of the part of fermentation broth that is added into the second and/or next bioreactor
(iii) Varying the time of step (i) and the amount of step (ii) until an optimal value is met using a dynamic process model; and
(iv) Adjusting the time in step (i) and/or the amount of the part of fermentation broth in step (ii) until the optimal value is met.

### DETAILED DESCRIPTION

The present invention relates to a method for cultivating a microorganism, comprising the steps of
(i) adding into a first bioreactor a medium and an inoculum comprising the microorganism and producing a fermentation broth in fed-batch culture; and
(ii) adding into a second bioreactor a second medium and part of the fermentation broth from step (i) and producing a second fermentation broth in a second fed-batch culture; and
(iii) adding into a next bioreactor a next medium and a part of the fermentation broth from step (ii), and producing a next fermentation broth in a next fed-batch culture, wherein a feed is introduced into the first, second and next bioreactor.

Surprisingly, it was found that when a bioreactor both receives (from a previous bioreactor) and donates (to a next bioreactor) a fermentation broth, an increased productivity of a method for cultivating microorganisms was found as compared to a method for cultivating microorganisms in fed-batch wherein the fermentation broth from a previous bioreactor is not used.

The method as disclosed herein is advantageously used in a fermentation plant comprising at least three, four, five, six, seven, eight, nine, ten, or even more bioreactors. The bioreactors in a method as disclosed herein may be deployed in all stages of the method of the invention. The same bioreactors may be used as a first, a second or any next bioreactor.

Producing a fermentation broth in a method according to the invention is performed as a fed-batch culture, which is known to a person skilled in the art. A fed-batch culture is defined herein as a fermentation process, wherein a medium comprising most of the nutrients for growing a microorganism and a microorganism are added at the start of the fermentation process, and wherein one or more nutrients, such a carbon source such as glucose, are fed to a bioreactor during fermentation or cultivation (the feed) and in which products remain in the bioreactor until the end of the fermentation process.

Each step, i.e. step (i), step (ii), step (iii) and any next step in the method as disclosed herein comprises producing a fermentation broth in fed-batch culture. The fed-batch culture in each step is usually continued until a bioreactor is filled with fermentation broth. At the end of a fed-batch culture a bioreactor is filled for 80% to 100% with fermentation broth, such as for 90% to 99% with fermentation broth.

The medium that is added to the first bioreactor in step (i) of the method of the invention may comprise any suitable nutrients, such as vitamins and any suitable nitrogen, phosphorous or other nutrients for cultivating the microorganism in the method of the invention. The medium may be a nutrient rich medium or a medium that has been depleted with certain nutrients, depending on the compound of interest that may be produced.

The medium that is added in a second bioreactor and a next bioreactor in step (ii), step (iii) and/or a next step in the method of the invention, i.e. the second medium and the next medium, may be a medium comprising the same nutrients as the medium that is added to the first bioreactor. The medium that is added into a second and/or next bioreactor may also comprise different or further nutrients compared to the medium that is added to the first bioreactor. In one preferred embodiment, the second medium and the next medium comprise the same nutrients as the medium that is added to the first bioreactor. In other words, the second medium and the next medium are the same medium as the one that is added to the first bioreactor.

Adding a medium into a bioreactor is performed by any suitable means, and usually is performed under sterile conditions known to a person skilled in the art. The medium that is added into a bioreactor in a method as disclosed herein is usually sterile. Working under sterile conditions avoids contamination by unwanted microorganisms. The medium in a method as disclosed herein comprises any suitable nutrients for cultivating the microorganism, such as a carbon source, for instance a sugar, and a nitrogen source such ammonia or urea, trace elements and vitamins. The medium in a fed-batch does not contain all nutrients for cultivating a microorganism. Usually the medium only contains a part of the carbon source, and another part is introduced with the feed. The medium is added to the bioreactor before or at the start of a fed-batch culture.

The first, second and any next bioreactor is sterile before adding a medium or the bioreactors are sterilized together with a medium, and subsequently an inoculum comprising a microorganism, and/or a fermentation broth is/are added into the bioreactor.

Producing a fermentation broth in fed-batch culture, further comprises introducing a feed into the first, second and next bioreactor. The feed, i.e. the first feed, the second feed and the next feed in step (i), step (ii) and step (iii), respectively, in a method as disclosed herein have a similar composition. The feed in step (i), step (ii) and step (iii) and any next step in the method of the invention comprises one or more suitable nutrients for cultivating the microorganism known to a person skilled in the art, for instance a carbon source such a glucose, fructose, maltose or sucrose. Usually the feed in the fed-batch culture comprises a nutrient that is not or only partially present in the medium that is added into the bioreactor.

The present disclosure relates to a method for cultivating a microorganism comprising the steps of
(i) adding into a first bioreactor a medium and an inoculum comprising the microorganism and producing a fermentation broth in fed-batch culture, wherein a first feed is introduced into the first bioreactor, and
(ii) adding into a second bioreactor a second medium and part of the fermentation broth from step (i) and producing a second fermentation broth in a second fed-batch culture, wherein a second feed is introduced into the second bioreactor, and
(iii) adding into a next bioreactor a next medium and a part of the fermentation broth from step (ii), and producing a next fermentation broth in a next fed-batch culture, wherein a next feed is introduced into the next bioreactor

The method as disclosed herein may further comprise a step of preparing an inoculum comprising the microorganism in an inoculum bioreactor before step (i). Preparing an inoculum of a microorganism can be performed by any suitable method in the art, and usually comprises growing the microorganism in a suitable inoculum medium in an inoculum bioreactor. An inoculum bioreactor is smaller than any of the bioreactors wherein a fermentation broth is produced. An inoculum bioreactor has a volume that is usually a factor five to fifteen times smaller than the bioreactor for producing a fermentation broth. The inoculum bioreactor usually has a volume of 1 to 100 m³, such as a volume of 2 to 50 m³, such as a volume of 5 to 20 m³.

In one embodiment, step (ii), step (iii) and/or any next step in a method of the invention further comprises adding an inoculum comprising the microorganism into the second and/or next bioreactor. Adding an inoculum may be performed at any suitable moment in step (ii), step (iii) and/or any next step. Preferably, adding an inoculum is performed before adding a part of a fermentation broth to bioreactor. It was found that adding an inoculum and a part of the fermentation broth of step (i), step (ii) and or any subsequent step into the second and/or next bioreactor can further increase the productivity for cultivating a microorganism in a method of the invention.

In one embodiment the method of the present invention further comprises a step (iv) which comprises repeating step (iii), wherein the part of the fermentation broth is from a previous step. The method as disclosed herein comprises repeating step (iii) as often as required or possible as long as the fermentation broth is not contaminated with undesirable microorganisms. Preferably, step (iii) is repeated such that the fermentation broth that is received from an earlier started fed-batch process has a composition that is similar to the composition of the fermentation broth that is donated to a next bioreactor.

Repeating step (iii) comprising introducing into a next bioreactor a medium and a part of the fermentation broth from a previous step, and optionally an inoculum comprising the microorganism, and producing a next fermentation broth may be performed from 1 to 100 times, for instance repeating step (iii) is performed 2 to 80 times, for instance 4 to 60 times, for instance 8 to 40 times.

It was found that repeating step (iii) in a method as disclosed herein is advantageously applied on an industrial scale, such as in a fermentation plant. An industrial scale method for cultivating microorganisms is defined herein that the bioreactors for producing a fermentation broth have a volume of 10 to 800 m³, or of 30 to 500 m³, preferably within of 50 to 450 m³, preferably of 100 to 400 m³.

Preferably, the fermentation broth that is taken from a first, second and/or next bioreactor is an amount that is equal to the part that is introduced into the second and/or next bioreactor.

The first, second and/or next bioreactor in a method or fermentation plant as disclosed herein have an equal volume, or a volume whereby the volume of any bioreactor is from 75% to 125% of the volume of any other bioreactor, such as the volume of any bioreactor is from 80% to 120%, such as from 85% to 115%, such as from 90% to 110%, such as from 95% to 105% of the volume any other bioreactor.

The first, second and/or a next bioreactor, in a method or fermentation plant as disclosed herein may have any suitable volume. The bioreactors may have a volume of 10 to 800 m³, or of 30 to 500 m³, preferably of 50 to 450 m³, preferably of 100 to 400 m³.

A next bioreactor in a method as disclosed herein is a bioreactor that is used after the second or any next bioreactor. A next bioreactor may be defined as the (n+1)^{th} bioreactor, wherein n is at least 2.

In one embodiment the method of the present invention further comprises removing a part of the fermentation broth from the first, second and/or next bioreactor, wherein the part of fermentation broth that is removed is added into the second and/or next bioreactor in step (ii), step (iii) and/or next step. The part that is removed from the first bioreactor has an equal volume as, or is equal to the part of fermentation broth that is added into the second bioreactor. Similarly, the part that is removed from the second bioreactor has an equal volume as, or is equal to the part of fermentation broth that is added into the next bioreactor. Likewise, the part that is removed from a next bioreactor has an equal volume as, or is equal to the part of fermentation broth that is added into the following next bioreactor.

In the method as disclosed herein any suitable part of the fermentation broth from the previous step may be added into a next bioreactor. Said part of the fermentation broth may be from 3 to 60% (w/w) of the fermentation broth of any of the previous step(s), for instance 5 to 50% (w/w), for instance 10 to 40% (w/w), for instance 12 to 30% (w/w) of the fermentation broth of any of the previous step(s).

The method of the present invention further comprises continuing producing a fermentation broth in fed-batch culture in step (i), step (ii), step (iii) and/or a next step after removing a part of the fermentation broth. Continuing producing a fermentation broth in a method of the present invention is performed according to methods known to a person skilled in the art, such as introducing a feed to the bioreactor. Continuing producing a fermentation broth in fed-batch culture is performed until the bioreactor is filled for 80% to 100% of the volume of the bioreactor, such as from 90% to 99% of the volume of a bioreactor.

In an embodiment, the fermentation broth in the first bioreactor is produced for 1 hour to 1 month before adding a part of the fermentation broth into the second bioreactor. For instance, the fermentation broth in the first bioreactor is produced for 2 hours to 20 days, or from 5 hours to 10 days, or from 10 hours to 5 days, or from 20 hours to 2 days before introducing a part of the fermentation broth into the next bioreactor.

In an embodiment, the fermentation broth in the second bioreactor is produced for 1 hour to 1 month before adding a part of the fermentation broth into a next bioreactor. For instance, the fermentation broth in the second bioreactor is produced for 2 hours to 20 days, or from 5 hours to 10 days, or from 10 hours to 5 days, or from 20 hours to 2 days before introducing a part of the fermentation broth into the next bioreactor.

In a similar embodiment, the fermentation broth in a next bioreactor is produced for 1 hour to 1 month before adding a part of the fermentation broth into a subsequent next bioreactor. For instance, the fermentation broth in the next bioreactor is produced for 2 hours to 20 days, or from 5 hours to 10 days, or from 10 hours to 5 days, or from 20 hours to 2 days before adding a part of the fermentation broth into the next bioreactor.

In the method for cultivating a microorganism the bioreactors are connected with means for transporting the fermentation broth between the bioreactors. Such means are known to a person skilled in the art, such as conduits and connecting means.

For the purpose of clarity, the method of the present invention is not a continuous cultivation process. In the method of the present invention, adding a part of the fermentation broth into a bioreactor (i.e. into the second or next bioreactor in step (ii), step (iii) and/or a next step) and/or removing a part of the fermentation broth from a reactor (i.e. from the first, second and/or next bioreactor) takes place intermittently (i.e. discontinuously). In other words, a bioreactor in the method of the present invention does not continuously receive a part of a fermentation broth from a previous bioreactor and/or does not continuously donate a part of a fermentation broth to a next bioreactor. In yet other words, adding a part of the fermentation broth into a bioreactor and/or removing a part of the fermentation broth from a bioreactor takes place in a limited period of time relative to the total time of producing a fermentation broth in fed-batch culture in said bioreactor. In one embodiment, adding into a bioreactor and/or removing from a bioreactor a part of a fermentation broth may take place in less than 10% of the total time of producing a fermentation broth in fed-batch culture in said bioreactor, preferably in less than 5% of the total time of producing a fermentation broth in fed-batch culture in said bioreactor, more preferably in less than 2% of the total time of producing a fermentation broth in fed-batch culture in said bioreactor.

The total time of producing a fermentation broth in fed-batch culture in a bioreactor (i.e. the total fermentation time) is calculated as the time duration between the time point of earliest addition of a microorganism in the bioreactor and the end time point of stopping the fed-batch culture to harvest the final fermentation broth from the bioreactor. The time point of earliest addition of a microorganism in the bioreactor may be the time point of adding an inoculum comprising the microorganism in the bioreactor and/or the time point of adding a part of a fermentation broth from a previous bioreactor. In an embodiment, the total fermentation time is between 8 hours and 50 days, preferably between 10 hours and 40 days, preferably between 10 hours and 35 days, more preferably between 10 hours and 20 days, even more preferably between 10 hours and 15 days, most preferably between 10 hours and 7 days.

In another embodiment, the method of the invention may be further expedited by adding additional inoculum broth from the microorganism to a first, second and/or a next bioreactor.

Preferably, the first, second and/or a next bioreactor is empty, and/or sterile before medium and/or an inoculum comprising the microorganism and/or a part of the fermentation broth of a previous step is introduced into the bioreactor.

The present invention also relates to a fermentation plant comprising at least three bioreactors wherein the bioreactors comprise means for transporting fermentation broth between bioreactors. The fermentation plant of the invention is suitable for carrying out the method as disclosed herein.

In an embodiment, the at least three bioreactors are production bioreactors, wherein the production bioreactors are connected with means for transporting fermentation broth between the bioreactors or production bioreactors. All bioreactors, or production bioreactors, may be connected with means for transporting fermentation broth between the bioreactors to all other bioreactors or production bioreactors. The means for transporting fermentation broth between the bioreactors or production bioreactors are conduits. Preferably, the present means for transporting fermentation broth between the bioreactors, or conduits, are situated in such a way that the inlet and/or outlet of the means for transporting fermentation broth allows the transport of fermentation broth between the bioreactors or production bioreactors. The inlet of the means for transporting fermentation broth is preferably situated at the underside or bottom of the bioreactor.

Cultivating a microorganism may be performed under any suitable conditions known to a person skilled in the art and depends on the microorganism that is cultivated in method of the present invention and the compound of interest that may be produced.

Any suitable microorganisms may be cultivated in a method as disclosed herein.

The microorganism may be selected from the group consisting of yeast, filamentous fungi, bacteria and algae.

A yeast may be a yeast belonging to the genus of Candida, Hansenula, Kluyveromyces, Pichia, Saccharomyces, Schizosaccharomyces, or Yarrowia. More preferably, the present yeast is Kluyveromyces lactis, Saccharomyces cerevisiae, Hansenula polymorpha, Yarrowia lipolytica or Pichia pastoris.

Filamentous fungal strains include, but are not limited to, strains of Acremonium, Agaricus, Aspergillus, Aureobasidium, Chrysosporium, Coprinus, Cryptococcus, Filibasidium, Fusarium, Humicola, Magnaporthe, Mucor, Myceliophthora, Neocallimastix, Neurospora, Paecilomyces, Penicillium, Piromyces, Panerochaete, Pleurotus, Schizophyllum, Talaromyces, Rasamsonia, Thermoascus, Thielavia, Tolypocladium, and Trichoderma.

Filamentous fungi may belong to a species of an Acremonium, Aspergillus, Chrysosporium, Myceliophthora, Penicillium, Talaromyces, Rasamsonia, Thielavia, Fusarium or Trichoderma genus, and most preferably a species of Aspergillus niger, Acremonium alabamense, Aspergillus awamori, Aspergillus foetidus, Aspergillus sojae, Aspergillus fumigatus, Talaromyces emersonii, Rasamsonia emersonii, Aspergillus oryzae, Chrysosporium lucknowense, Fusarium oxysporum, Myceliophthora thermophila, Trichoderma reesei, Thielavia terrestris or Penicillium chrysogenum. A more preferred filamentous fungi belongs to the genus Aspergillus, more preferably the filamentous fungi belongs to the species Aspergillus niger or is Aspergillus niger.

The term “bacteria” includes both Gram-negative and Gram-positive microorganisms. Suitable bacteria may be selected from e.g. Escherichia, Anabaena, Caulobactert, Gluconobacter, Rhodobacter, Pseudomonas, Paracoccus, Bacillus, Brevbacterium, Corynebacterium, Rhizobium (Sinorhizobium), Flavobacterium, Klebsiella, Enterobacter, Lactobacillus, Lactococcus, Methylobacterium, Staphylococcus, Streptomyces, Actinomycetes, Xanthomonas or Sphingomonas. Preferably, the bacterial cell is selected from the group consisting of B. subtilis, B. amyloliquefaciens, B. licheniformis, B. puntis, B. megaterium, B. halodurans, B. pumilus, G. oxydans, Caulobactert crescentus CB 15, Methylobacterium extorquens, Rhodobacter sphaeroides, Pseudomonas zeaxanthinifaciens, Paracoccus denitrificans, E. coli, C. glutamicum, Staphylococcus camosus, Streptomyces lividans, Sinorhizobium melioti and Rhizobium radiobacter.

Algae as used herein may be glaucophytes, rhodoplasts, chloroplasts. More preferably the present algae are heterotrophic algae, more preferably heterotrophic algae like Chlorella, Nannochloropsys, Nitzschia, Thraustochytrium, Aurantiochytrium, or Schizochytrium.

Cultivating a microorganism in a method as disclosed herein may further comprise producing a compound of interest.

The microorganism in a method as disclosed herein may comprise at least one polynucleotide coding for a compound of interest or at least one polynucleotide coding for a compound involved in the production of a compound of interest by the cell.

The polynucleotide coding for the compound of interest or coding for a compound involved in the production of the compound of interest may encode an enzyme involved in the synthesis of a primary or secondary metabolite, such as organic acids, alcohols, lipids, carotenoids, beta-lactam, antibiotics, and vitamins.

The compound of interest can be any biological compound. The biological compound may be biomass or a biopolymer or metabolite. The biological compound may be encoded by a single polynucleotide or a series of polynucleotides composing a biosynthetic or metabolic pathway or may be the direct result of the product of a single polynucleotide or products of a series of polynucleotides. The biological compound may be native to the host cell or heterologous.

The term “heterologous biological compound” is defined herein as a biological compound which is not native to the cell; or a native biological compound in which structural modifications have been made to alter the native biological compound.

The term “biopolymer” is defined herein as a chain (or polymer) of identical, similar, or dissimilar subunits (monomers). The biopolymer may be any biopolymer, for instance a polypeptide or a polysaccharide.

The biopolymer may be a polypeptide. The polypeptide may be any polypeptide having a biological activity of interest. The term “polypeptide” is not meant herein to refer to a specific length of the encoded product and, therefore, encompasses peptides, oligopeptides, and proteins. Polypeptides further include naturally occurring allelic and engineered variations of the above-mentioned polypeptides and hybrid polypeptides. The polypeptide may be native or may be heterologous to the host cell. The polypeptide may be a collagen or gelatin, or a variant or hybrid thereof. The polypeptide may be an antibody or parts thereof, an antigen, a clotting factor, an enzyme, a hormone or a hormone variant, a receptor or parts thereof, a regulatory protein, a structural protein, a reporter, or a transport protein, protein involved in secretion process, protein involved in folding process, chaperone, peptide amino acid transporter, glycosylation factor, transcription factor, synthetic peptide or oligopeptide, intracellular protein.

The intracellular protein may be an enzyme such as, a protease, ceramidases, epoxide hydrolase, aminopeptidase, acylases, aldolase, hydroxylase, aminopeptidase, lipase.

The polypeptide may also be an enzyme secreted extracellularly. Such enzymes may belong to the groups of oxidoreductase, transferase, hydrolase, lyase, isomerase, ligase, catalase, cellulase, chitinase, cutinase, deoxyribonuclease, dextranase, esterase. The enzyme may be a carbohydrase, e.g. cellulases such as endoglucanases, β-glucanases, cellobiohydrolases or β-glucosidases, hemicellulases or pectinolytic enzymes such as xylanases, xylosidases, mannanases, galactanases, galactosidases, pectin methyl esterases, pectin lyases, pectate lyases, endo polygalacturonases, exopolygalacturonases rhamnogalacturonases, arabanases, arabinofuranosidases, arabinoxylan hydrolases, galacturonases, lyases, or amylolytic enzymes; hydrolase, isomerase, or ligase, phosphatases such as phytases, esterases such as lipases, proteolytic enzymes, oxidoreductases such as oxidases, transferases, or isomerases. The enzyme may be a phytase. The enzyme may be an aminopeptidase, asparaginase, amylase, a maltogenic amylase, carbohydrase, carboxypeptidase, endo-protease, metallo-protease, serine-protease catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, protein deaminase, invertase, laccase, lipase, mannosidase, mutanase, oxidase, pectinolytic enzyme, peroxidase, phospholipase, galactolipase, chlorophyllase, polyphenoloxidase, ribonuclease, transglutaminase, or glucose oxidase, hexose oxidase or monooxygenase.

The biopolymer may be a polysaccharide. The polysaccharide may be any polysaccharide, including, but not limited to, a mucopolysaccharide (e. g., heparin and hyaluronic acid) and nitrogen-containing polysaccharide (e.g., chitin). In a more preferred option, the polysaccharide is hyaluronic acid. In another preferred option, the polysaccharide is a hydrocolloid, e.g. xanthan, gellan, pectin, welan or another polysaccharide.

The term “metabolite” encompasses both primary and secondary metabolites; the metabolite may be any metabolite. Preferred metabolites are citric acid, gluconic acid, adipic acid, fumaric acid, itaconic acid and succinic acid.

The primary metabolite may be, but is not limited to, an amino acid, fatty acid, triacylglycerol, nucleoside, nucleotide, sugar, triglyceride, or vitamin. For example, vitamin A, B2, C, D or E.

The secondary metabolite may be, but is not limited to, an alkaloid, coumarin, flavonoid, polyketide, quinine, steroid, peptide, or terpene. The secondary metabolite may be an antibiotic, antifeedant, attractant, bacteriocide, fungicide, hormone, insecticide, or rodenticide. Preferred antibiotics are cephalosporins and beta-lactams. Other preferred metabolites are exo-metabolites. Examples of exo-metabolites are Aurasperone B, Funalenone, Kotanin, Nigragillin, Orlandin, Other naphtho-γ-pyrones, Pyranonigrin A, Tensidol B, Fumonisin B2 and Ochratoxin A.

The present invention also relates to a process for controlling the method of the invention, comprising the steps of
(i) Determining a time the fermentation broth is present in the first, second and/or next bioreactor
(ii) Determining an amount of the part of fermentation broth that is added into the second and/or next bioreactor
(iii) Varying the time of step (i) and the amount of step (ii) until an optimal value is met using a dynamic process model; and
(iv) Adjusting the time in step (i) and/or the amount of the part of fermentation broth in step (ii) until the optimal value of step (iii) is met.

The time the fermentation broth is present in the first, second and/or next bioreactor is the duration for producing a fermentation broth as defined herein above.

The amount of the fermentation broth that is added into the second or next bioreactor can be any suitable amount. The amount of fermentation broth can be 3 to 60% (w/w) of the fermentation broth of any of the previous step(s), for instance 5 to 50% (w/w), for instance 10 to 40% (w/w), for instance 12 to 30% (w/w) of the fermentation broth of a previous step.

Varying the time the fermentation broth is present in the first, second and/or next bioreactor and/or the amount of fermentation broth in step (ii) may be performed using a dynamic model known in the art. A dynamic process model is a mathematical description of the microbial substrate consumption, biomass growth and product formation kinetics in a fermentation process, that are combined with the feeds into the fermentation process to give differential equations that describe the changes of the amounts of nutrients, biomass, product and bioproducts that are present in the bioreactor over time. A dynamic process model may for instance be a model disclosed in Gernaey K., et al (2010) Trends in Biotechnology 28, p. 346-354, or in Kroll (P. et al. (2017) Pharm. Res. 34, p. 2596-2613, and can be implemented in Matlab software (The Mathworks, Natick, MA, USA).

An optimal value in the process for controlling the method for cultivating a microorganism as disclosed herein, may for instance be the optimal productivity of the method of cultivating a microorganism of the invention, for instance the optimal productivity of a polypeptide or a compound of interest.

The invention also relates to a computer implemented method for carrying out the process for controlling the method of the present invention.

### FIGURES

FIG. 1. Simulated broth weight versus fermentation time of example 1 (dotted line) and example 2 (solid line)

FIG. 2. Simulated glucose concentration in broth versus fermentation time of example 1 (dotted line) and example 2 (solid line)

FIG. 3. Simulated ammonia concentration in broth versus fermentation time of example 1 (dotted line) and example 2 (solid line)

FIG. 4. Simulated biomass concentration in broth versus fermentation time of example 1 (dotted line) and example 2 (solid line)

FIG. 5. Simulated protein product concentration versus fermentation time of example 1 (dotted line) and example 2 (solid line)

FIG. 6. Simulated oxygen uptake rate versus fermentation time of example 1 (dotted line) and example 2 (solid line)

FIG. 7. Simulated carbon dioxide production rate versus fermentation time of example 1 (dotted line) and example 2 (solid line)

FIG. 8. Simulated glucose feed rate versus fermentation time of example 1 (dotted line) and example 2 (solid line)

FIG. 9. Simulated yield of product on sugar substrate versus fermentation time of example 1 (dotted line) and example 2 (solid line)

FIG. 10. Simulated economical productivity versus fermentation time of example 1 (dotted line) and example 2 (solid line)

FIG. 11. Simulated broth weight versus fermentation time of example 1 (dotted line) and example 3 (solid line)

FIG. 12. Simulated glucose concentration in broth versus fermentation time of example 1 (dotted line) and example 3 (solid line)

FIG. 13. Simulated ammonia concentration in broth versus fermentation time of example 1 (dotted line) and example 3 (solid line)

FIG. 14. Simulated biomass concentration in broth versus fermentation time of example 1 (dotted line) and example 3 (solid line)

FIG. 15. Simulated protein product concentration versus fermentation time of example 1 (dotted line) and example 3 (solid line)

FIG. 16. Simulated oxygen uptake rate versus fermentation time of example 1 (dotted line) and example 3 (solid line)

FIG. 17. Simulated carbon dioxide production rate versus fermentation time of example 1 (dotted line) and example 3 (solid line)

FIG. 18. Simulated glucose feed rate versus fermentation time of example 1 (dotted line) and example 3 (solid line)

FIG. 19. Simulated yield of product on sugar substrate versus fermentation time of example 1 (dotted line) and example 3 (solid line)

FIG. 20. Simulated economical productivity versus fermentation time of example 1 (dotted line) and example 3 (solid line)

FIG. 21. Simulated broth weight versus fermentation time of example 1 (dotted line) and example 4 (solid line)

FIG. 22. Simulated glucose concentration in broth versus fermentation time of example 1 (dotted line) and example 4 (solid line)

FIG. 23. Simulated ammonia concentration in broth versus fermentation time of example 1 (dotted line) and example 4 (solid line)

FIG. 24. Simulated biomass concentration in broth versus fermentation time of example 1 (dotted line) and example 4 (solid line)

FIG. 25. Simulated protein product concentration versus fermentation time of example 1 (dotted line) and example 4 (solid line)

FIG. 26. Simulated oxygen uptake rate versus fermentation time of example 1 (dotted line) and example 4 (solid line)

FIG. 27. Simulated carbon dioxide production rate versus fermentation time of example 1 (dotted line) and example 4 (solid line)

FIG. 28. Simulated glucose feed rate versus fermentation time of example 1 (dotted line) and example 4 (solid line)

FIG. 29. Simulated yield of product on sugar substrate versus fermentation time of example 1 (dotted line) and example 4 (solid line)

FIG. 30. Simulated economical productivity versus fermentation time of example 1 (dotted line) and example 4 (solid line)

### EXAMPLES

### Reference Example 1

An aerobic, fed-batch fermentation process is simulated in which biomass grows and produces a protein product in a batch medium that contains an excess of all nutrients that are required for growth and product formation during the entire process, except for the carbon source glucose and the nitrogen source ammonia of which only a minor part is provided in the batch medium.

The fed-batch process starts upon inoculation with an amount of biomass containing broth from a seed bioreactor, with an initial batch phase during which the biomass grows exponentially on the nutrients that are present in the batch medium. The batch phase ends when the glucose in the medium is depleted, and ammonia is still present in excess. At that moment the feeding phase starts.

During the feeding phase, ammonia is fed as a pH titrant such that the ammonia concentration never limits growth nor product formation. Glucose is fed as a solution in water, at a feeding rate that limits the glucose uptake rate and thereby determines the growth and product formation rates during the larger part of this phase. The glucose feeding rate is a linear function of the total amount of broth in the bioreactor.

During the process, oxygen is provided to, and carbon dioxide is removed from the broth by an airflow that is sparged into the fermentation broth in the bioreactor. The airflow and the mass transfer rates of oxygen and carbon dioxide between the gas phase and the fermentation broth are controlled such that oxygen is not or hardly limiting, and carbon dioxide is not or hardly inhibiting biomass growth nor protein product formation.

The gross volume of the bioreactor vessel is 100 m³. The initial broth weight including the inoculum is 40 tons. The fed-batch process is stopped at 136 h when the broth weight equals 80 tons, which equals 90-95 m³ of gassed broth volume. For the productivity calculation it is assumed that the turn-around time, needed to empty, clean and sterilize the bioreactor in between two subsequent fed-batch processes equals 12 h.

The growth and protein production of the biomass are described by a qₚ-μ correlation (qₚ=the biomass specific protein production rate; p=the biomass specific growth rate) which shows that the biomass specific protein production rate equals zero both when the biomass does not grow, and when the biomass grows at its maximum growth rate. The biomass specific protein production rate reaches a maximum value at a biomass specific growth rate in between zero and the maximum growth rate.

The simulation of the fermentation process is carried out using Matlab software (The Mathworks, Natick, MA, USA).

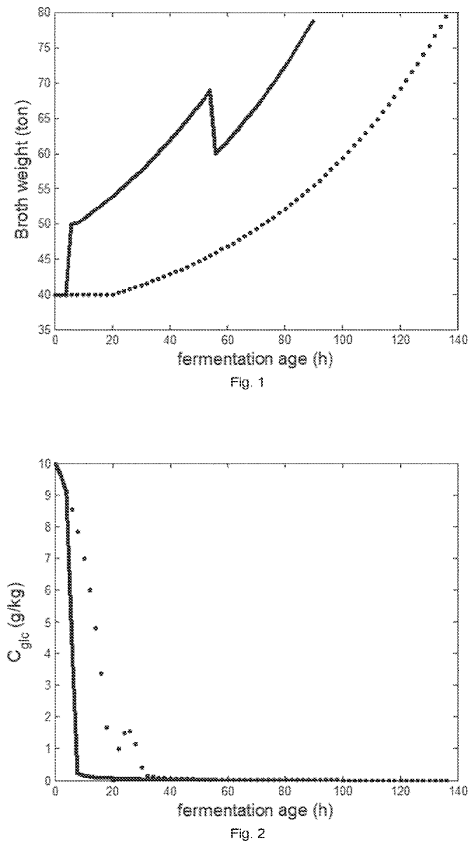

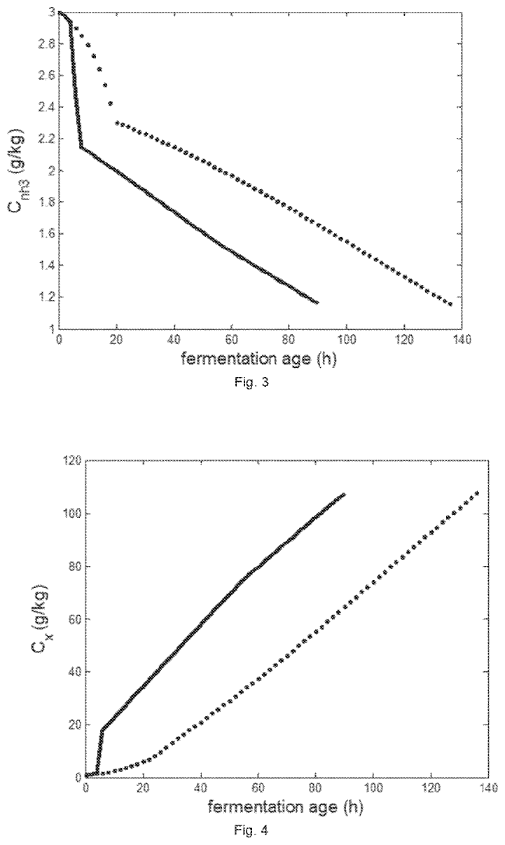

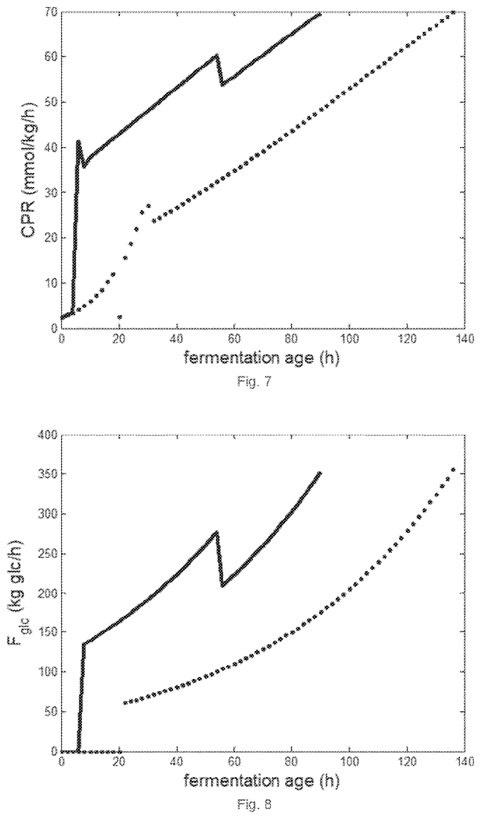

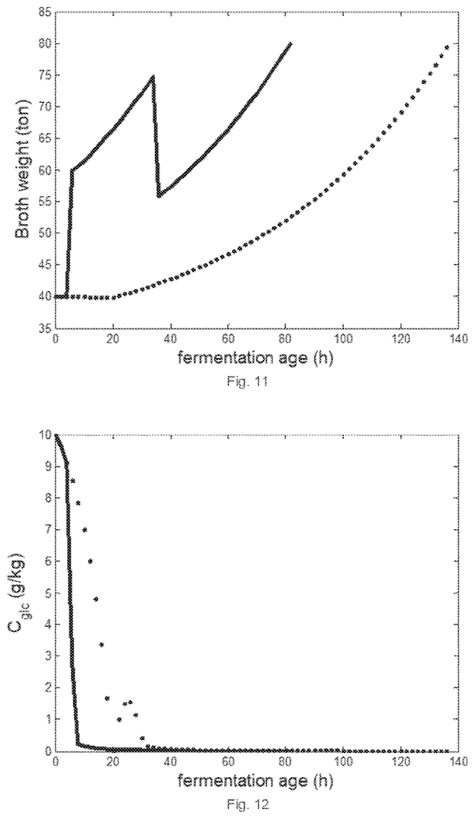

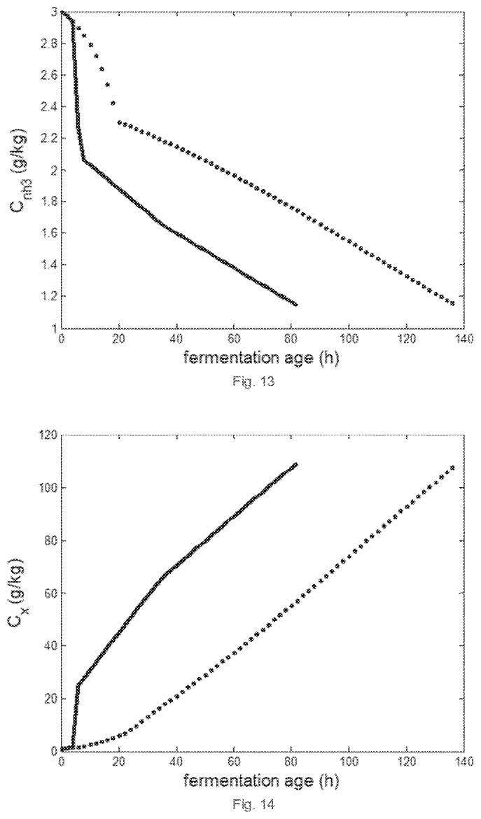

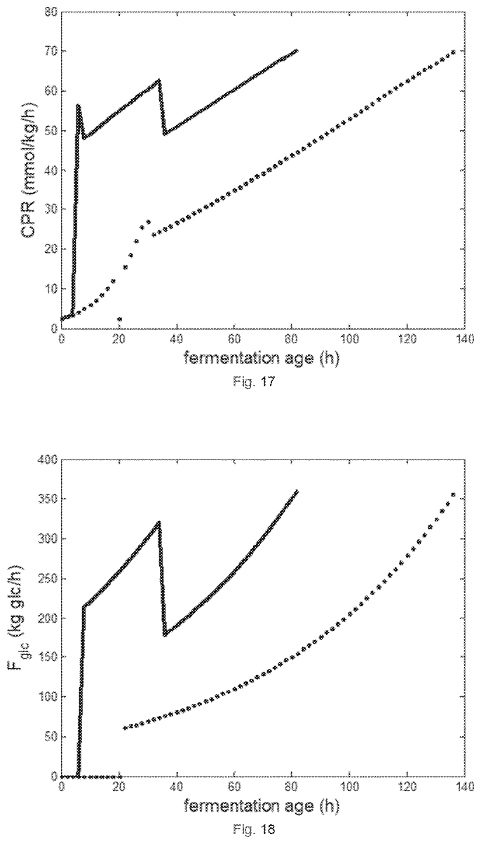

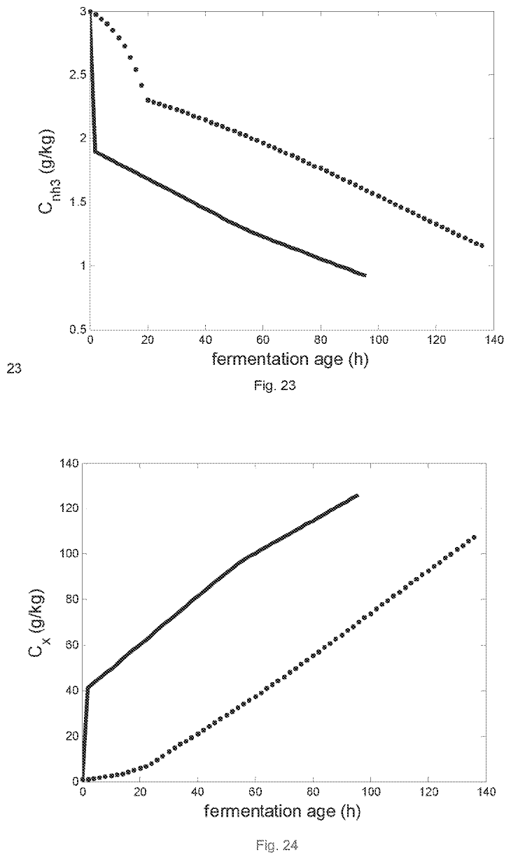

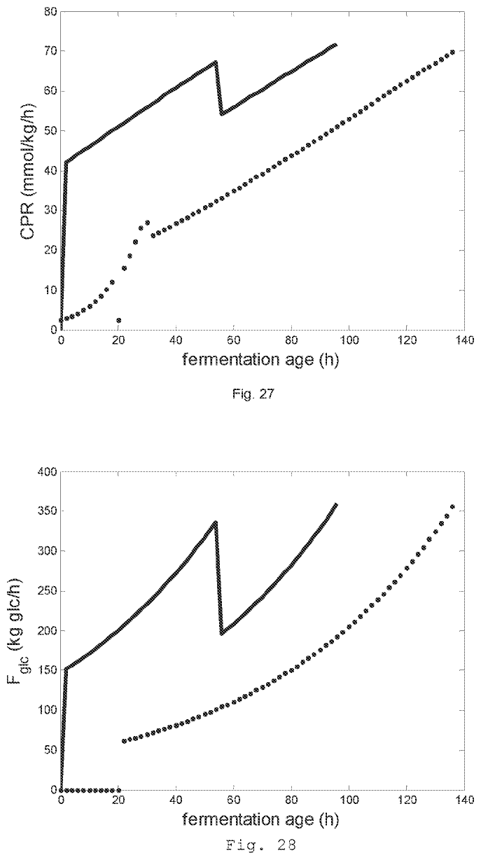

The results of the simulation are given as the dotted lines in FIGS. 1 to 10, and FIGS. 11 to 20, and FIGS. 21 to 30.

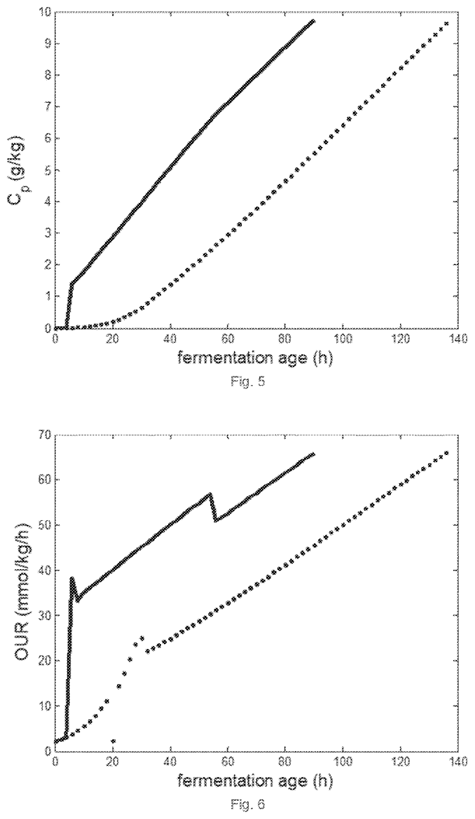

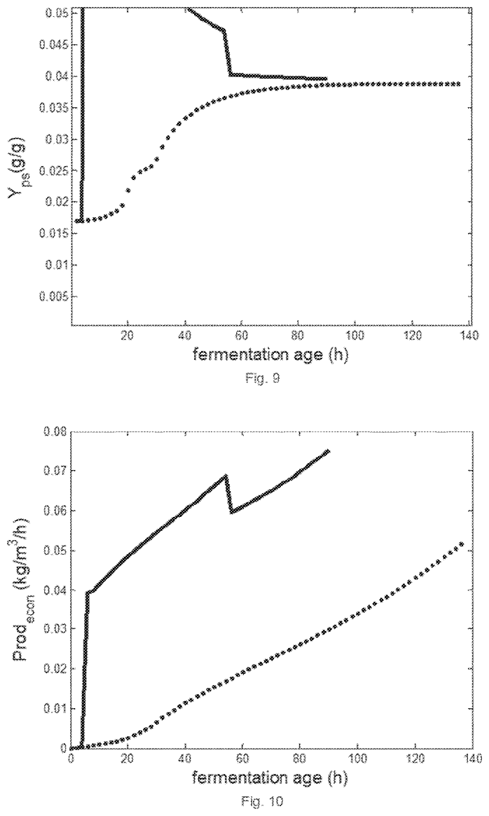

The performance of the fermentation process is judged by three key performance indicators:
the protein product concentration at the end of the fermentation, which equals 9.6 g product/kg broth, (FIGS. 5, 15 and 25)
the yield of protein product on sugar substrate at the end of the fermentation, which equals 0.039 g product/g glucose, (FIG. 9, 19, 29)
the economical productivity at the end of the fermentation, which equals 0.052 kg product/m³ total (gross) bioreactor volume/total fermentation time in the simulated bioreactor (including turn-around time). (FIGS. 10, 20 and 30)

### Example 2

The fed-batch fermentation process in this example is identical to example 1, with the exception that after 5 h of fermentation time, 10 tons of broth is transferred from an earlier started fed-batch process that has at that same moment a fermentation age of 55 h, to the simulated bioreactor. The broth transfer rate equals 40 tons/h.

Subsequently, when the simulated bioreactor itself has reached a fermentation age of 55 h, 10 tons of broth is withdrawn from this bioreactor. This broth is transferred to a next bioreactor that has at that same moment a fermentation age of 5 h. Again, the broth transfer rate equals 40 tons/h. The fed-batch process is stopped at 90 h when the broth weight equals 80 tons.

The earlier started fed-batch process from which broth was transferred to the simulated bioreactor had itself also received 10 tons of broth from a yet earlier started bioreactor at a fermentation age of 5 h. This latter step had happened recursively for at least 5 more times.

The results of the simulation are given as the solid lines in FIGS. 1 to 10. The performance of the fermentation process is judged by three key performance indicators:
the protein product concentration at the end of the fermentation, which equals 9.7 g product/kg broth, (FIG. 5)
the yield of protein product on sugar substrate at the end of the fermentation, which equals 0.040 g product/g glucose (FIG. 9),
the economical productivity at the end of the fermentation, which equals 0.075 kg product/m³ gross bioreactor volume/total fermentation time in the simulated bioreactor (including turn-around time). (FIG. 10)

### Example 3

The fed-batch fermentation process in this example is identical to example 1, with the exception that after 5 h of fermentation time, 20 tons of broth is transferred from an earlier started fed-batch process that has at that same moment a fermentation age of 35 h, to the simulated bioreactor. The broth transfer rate equals 40 tons/h.

Subsequently, when the simulated bioreactor itself has reached a fermentation age of 35 h, 20 tons of broth is withdrawn from this bioreactor. This broth is transferred to a next bioreactor that has at that same moment a fermentation age of 5 h. Again, the broth transfer rate equals 40 tons/h. The fed-batch process is stopped at 82 h when the broth weight equals 80 tons. The earlier started fed-batch process from which broth was transferred to the simulated bioreactor had itself also received 20 tons of broth from a yet earlier started bioreactor at a fermentation age of 5 h. This latter step had happened recursively for at least 5 more times.

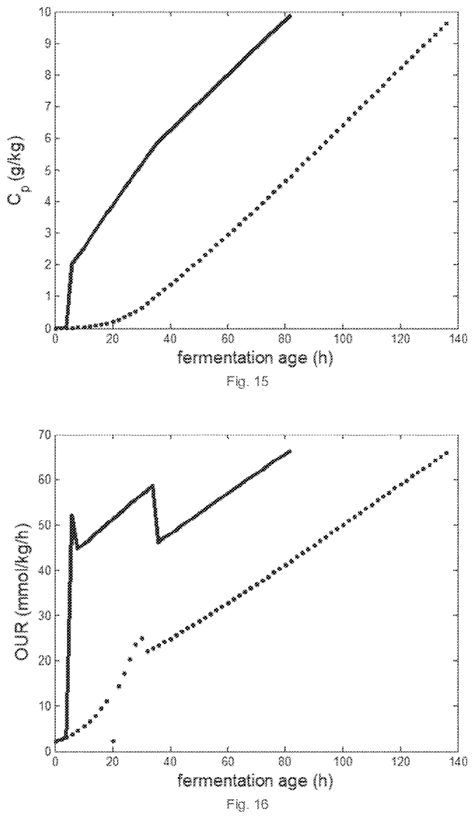

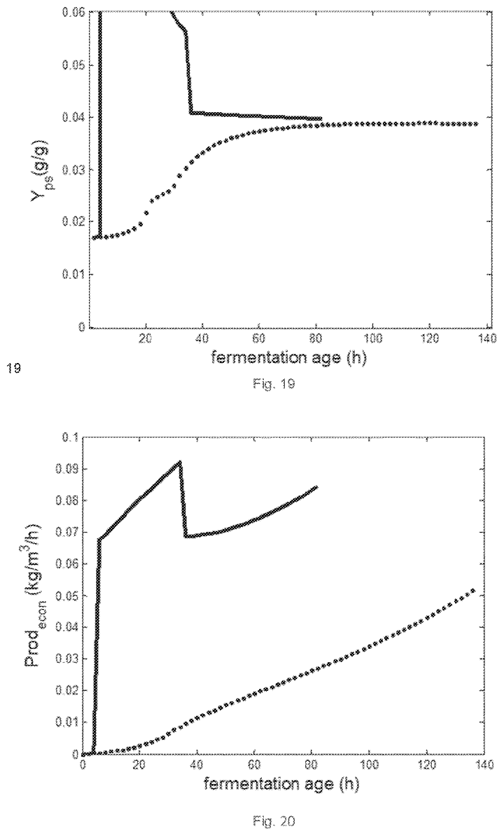

The results of the simulation are given as the solid lines in FIGS. 11 to 20. The performance of the fermentation process is judged by three key performance indicators:
the protein product concentration at the end of the fermentation, which equals 9.9 g product/kg broth, (FIG. 15)
the yield of protein product on sugar substrate at the end of the fermentation, which equals 0.040 g product/g glucose, (FIG. 19)
the economical productivity at the end of the fermentation, which equals 0.084 kg product/m gross bioreactor volume/total fermentation time in the simulated bioreactor (including turn-around time). (FIG. 20)

### Example 4

This example is identical to example 1, with the exception that no inoculum from a seed fermenter is added to the simulated bioreactor, as a result of which the initial broth weight is 32 tons instead of 40 tons. Another difference with example 1 is that after 0 h of fermentation time 20 tons of broth is transferred from an earlier started fed-batch process that has at that same moment a fermentation age of 55 h to the simulated bioreactor. The broth transfer rate equals 40 tons/h. Additionally, when the simulated bioreactor itself has reached a fermentation age of 55 h, 20 tons of broth is withdrawn from this bioreactor. This broth is transferred to a next bioreactor that has at that same moment a fermentation age of 0 h. The broth transfer rate equals 40 tons/h. The fed-batch process is stopped at 97 h when the broth weight equals 80 tons.

The earlier started fed-batch process from which broth was transferred to the simulated bioreactor had itself also received 20 tons of broth from a yet earlier started bioreactor at a fermentation age of 0 h. The same had happened recursively for at least 5 more times.

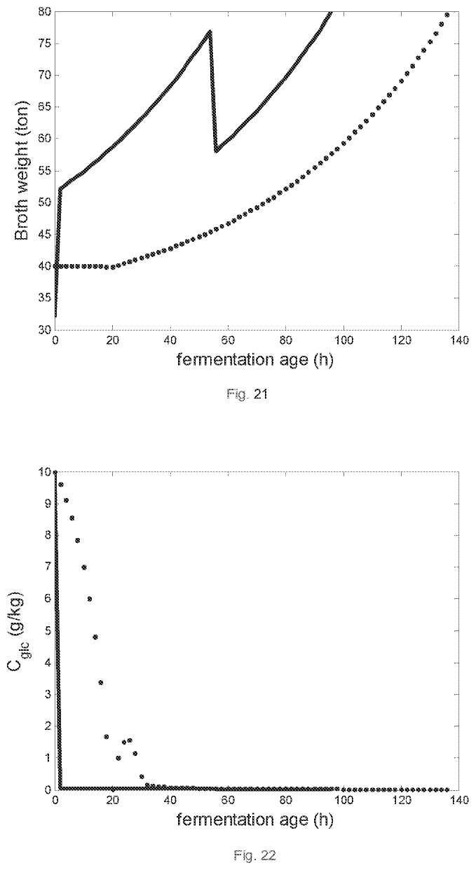

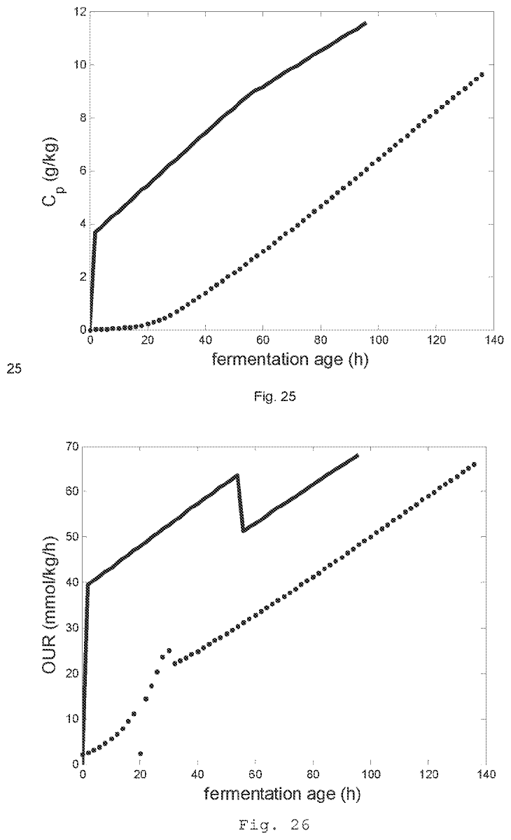

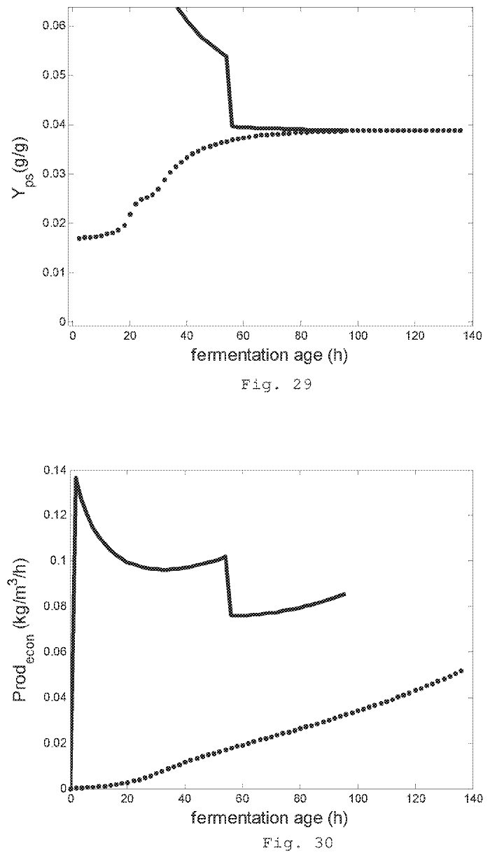

The results of the simulation are given as the solid lines in FIGS. 21 to 30. The performance of the fermentation process is judged by three key performance indicators:
the protein product concentration at the end of the fermentation, which equals 11.2 g product/kg broth (FIG. 25),
the yield of protein product on sugar substrate at the end of the fermentation, which equals 0.039 g product/g glucose (FIG. 29),
the economical productivity at the end of the fermentation, which equals 0.078 kg product/m³ gross bioreactor volume/total fermentation time in the simulated bioreactor (including turn-around time) (FIG. 30).

The results of Examples 2 to 4 surprisingly show that using part of a fermentation broth of a fed-batch culture as an inoculum into a next fed-batch fermentation culture and subsequently using part of the fermentation broth of the next fed-batch culture as an inoculum into another subsequent fed-batch fermentation culture, increases the productivity of the fed-batch fermentation process as compared to a fed-batch fermentation process wherein fermentation broth of a fed-batch culture is not used as an inoculum for a next fed-batch culture.

1. A method for cultivating a microorganism, comprising
(i) adding into a first bioreactor a medium and an inoculum comprising the microorganism and producing a fermentation broth in fed-batch culture; and
(ii) adding into a second bioreactor a second medium and part of the fermentation broth from (i) and producing a second fermentation broth in a second fed-batch culture; and
(iii) adding into a next bioreactor a next medium and a part of the fermentation broth from (ii), and producing a next fermentation broth in a next fed-batch culture, wherein a feed is introduced into the first, second and next bioreactor.

2. The method according to claim 1, wherein (ii) and/or (iii) further comprise(s) adding an inoculum comprising the microorganism into the second and/or next bioreactor.

3. The method according to claim 1, further comprising preparing an inoculum comprising the microorganism in an inoculum bioreactor before (i), (ii) and/or (iii).

4. The method according to claim 1, further comprising (iv) comprising repeating (iii), wherein the part of the fermentation broth is from i ii and/or iii.

5. The method according to claim 4, wherein repeating (iii) is performed 1 to 100 times.

6. The method according to any on of the claim 1 wherein in (ii), (iii) and/or thereafter, said part of the fermentation broth is from 3 to 60% (w/w) of the fermentation broth from previously.

7. The method according to claim 1, further comprising removing a part of the fermentation broth from the first, second and/or next bioreactor, and adding the part of fermentation broth that is removed into the second and/or next bioreactor in (ii), (iii) and/or thereafter.

8. The method according to claim 1, further comprising continuing producing a fermentation broth in fed-batch culture in (i), (ii), (iii) and/or thereafter, after removing a part of the fermentation broth from the first, second and/or next bioreactor and/or adding the part of the fermentation broth into the second or next bioreactor in (ii), (iii) and/or thereafter.

9. The method according to claim 1, wherein the fermentation broth is produced for 2 hours to 20 days before adding a part of the fermentation broth into the second and/or next bioreactor.

10. The method according to claim 1, wherein cultivating the microorganism comprises producing a compound of interest.

11. The method according to claim 1, wherein adding part of the fermentation broth into the second or next bioreactor in (ii), (iii) and/or thereafter takes place intermittently.

12. A fermentation plant for carrying out a method according to claim 1, comprising at least three bioreactors, wherein the bioreactors comprise means for transporting a fermentation broth between the bioreactors.

13. The fermentation plant according to claim 12, wherein all bioreactors are connected with means for transporting a fermentation broth between bioreactors to all other bioreactors.

14. The fermentation plant according to claim 12, or the method used therein wherein the first, second and/or next bioreactor has a volume from 10 to 500 m³.

15. A process for controlling the method according to claim 1, comprising
(i) Determining a time the fermentation broth is present in the first, second and/or next bioreactor
(ii) Determining an amount of the part of fermentation broth that is added into the second and/or next bioreactor
(iii) Varying the time of (i) and the amount of (ii) until an optimal value is met using a dynamic process model; and
Adjusting the time in (i) and/or the amount of the part of fermentation broth in (ii) until the optimal value is met.
